(19) United States
(12) Patent Application Publication
(10) Pub. No.: US 2009/0005721 A1
(43) Pub. Date: Jan. 1, 2009

(54) PACKAGED IONTOPHORESIS SYSTEM

(75) Inventors: Mizuo Nakayama, Tokyo (JP); Takehiko Matsumura, Kanagawa (JP); Tsutomu Shibata, Chiba (JP); Akihiro Ryuka, Tokyo (JP)

Correspondence Address: SEED INTELLECTUAL PROPERTY LAW GROUP PLLC, 701 FIFTH AVE, SUITE 5400, SEATTLE, WA, 98104, US

(73) Assignee: TTI ELLEBEAU, INC., Tokyo (JP)

(21) Appl. No.: 12/135,901

(22) Filed: Jun. 9, 2008

Related U.S. Application Data
(63) Continuation of application No. PCT/JP2006/324182, filed on Dec. 4, 2006.

(30) Foreign Application Priority Data
Dec. 9, 2005 (JP) 2005-356431

(51) Int. Cl. A61N 1/30

(52) U.S. Cl. 604/20

(57) ABSTRACT

Provided is a packaged iontophoresis system 15 including: an iontophoresis system 10 including an apparatus body 50 for transdermally delivering an ionized drug by iontophoresis, and a connection part 70 extended from the apparatus body 50 and connected to the apparatus body 50 and a power source 60, the power source 60 supplying power to the apparatus body 50; a package material 80 hermetically packaging the apparatus body 50 and a portion of the connection part 70, by at least a part thereof being welded to the connection part 70; and a cutting guide 400 guiding a cutting line of the package material 80 so that the part of the package material 80 welded to the connection part 70 remains at the connection part 70 when the package material 80 is opened.

### CROSS REFERENCE TO RELATED APPLICATION

This is a continuation application of PCT/JP2006/324182 filed on Dec. 4, 2006 which claims priority from a Japanese Patent Application No. 2005-356431 filed on Dec. 9, 2005, the contents of which are incorporated herein by reference.

### BACKGROUND

1. Technical Field

The present invention relates to a packaged iontophoresis system. In particular, the present invention relates to a packaged iontophoresis system hermetically packaging an iontophoresis system for transdermally delivering drug ions by iontophoresis.

2. Related Art

Japanese Patent Application Publication No. 2000-229128 discloses an iontophoresis system for transdermally delivering drug ions to biologic surfaces, such as skin or mucous membranes, of a predetermined part of a human or animal body by iontophoresis. Hereinafter, such skin and mucous membranes are collectively referred to as “skin”.

The iontophoresis system disclosed in Japanese Patent Application Publication No. 2000-229128 attempts to solve such problems as a decrease amount of ionic drug or drug-dissolving solvent from a container of the iontophoresis system due to volatilization, and corrosion of a power source such as a battery attributable to the vaporized drug solutions due to the volatilization, during storage prior to application of the drug ions to a patient. A possible solution thereof is to fix the power source of the apparatus body of the iontophoresis system to the package material to hermetically sealing them. In this case, however, the package material will be welded to a connection part between the power source and the apparatus body. This has caused a problem that the surface of the connection part is peeled off with the package material when opening the package material.

### SUMMARY

In view of this, it is an object of one aspect of the present invention to provide a packaged iontophoresis system capable of addressing the foregoing problems. This object may be achieved by combinations of features described in the independent claims. The dependent claims define further advantageous and concrete embodiments of the present invention.

According to one exemplary packaged iontophoresis system based on an aspect of the innovation herein, a packaged iontophoresis system includes an apparatus body for transdermally delivering an ionized drug by iontophoresis, and an iontophoresis device having a connection part extended from the apparatus body which is connected to a power source supplying power to the apparatus body; a package material hermetically packaging the apparatus body and a portion of the connection part by at least a portion thereof being fixed to the connection part; and a cutting guide for guiding a cutting line of the package material so that the part of the package material fixed to the connection part remains with the connection part when the package material is opened and removed. Accordingly, the package material can be opened to be removed without damaging the wiring of the connection part or the like.

In the above described packaged iontophoresis system, the cutting guide part may also include a groove in a thickness direction and provided between the portion thereof that is fixed to the connection part and the other portion thereof. Accordingly, when opening and removing the package material, the cutting guide is able to more assuredly guide the cutting line so that the part fixed to the connection part will remain at the connection part.

In the above described packaged iontophoresis system, the cutting guide part may also include a notch provided in the vicinity of the fixed part to the connection part. Accordingly, when opening and removing the package material the cutting guide is able to more assuredly guide the cutting line so that the part fixed to the connection part will remain at the connection part.

In the above described package material, preferably, the strength of the part fixed to the connection part is stronger than any other part of the package material. Accordingly, the package material can be opened to be removed, without damaging wiring in the connection part or the like.

The above described iontophoresis system may also have an apparatus body that includes: an operation electrode structure including a first electrode member and a drug solution retainer, the first electrode member being electrically connected to a first conductivity type terminal of the power source, the first conductivity type being the same as a conductivity type of the ionized drug, the drug solution retainer retaining a drug solution containing the drug and being provided in an electric field created by the first electrode member; and a non-operation electrode structure electrically connected to a second conductivity type terminal of the power source, the second conductivity type being opposite to the first conductivity type. Accordingly, iontophoresis can be applied to any drug solution containing a drug, without special preparations.

It is further possible to arrange the iontophoresis system so that the operation electrode structure includes: a first electrolyte retainer electrically connected to the first electrode member and retaining an electrolyte; an ion exchange membrane of the second conductivity type sandwiching the first electrolyte retainer with the first electrode member, and selectively transmitting ions of the second conductivity type; and an ion exchange membrane of the first conductivity type sandwiching the drug solution retainer with the ion exchange membrane of the second conductivity type, and selectively transmitting ions of the first conductivity type. Accordingly, not only can the skin to be in contact with the operation electrode structure be prevented from burns and inflammation, but also the drug ions can be applied while stably supplying electricity to the skin. This enables the drug ions to be applied to the living organism securely and efficiently.

It is further possible to arrange the iontophoretic system so that the non-operation electrode structure includes: a second electrode member electrically connected to the terminal of the second conductivity type of the power source; a second electrolyte retainer electrically connected to the second electrode member and retaining an electrolyte; an ion exchange membrane of the first conductivity type sandwiching the second electrolyte retainer with the second electrode member, and selectively transmitting ions having an electric polarity different from an electric polarity of the second electrode member; a third electrolyte retainer provided in an opposite side of the second electrolyte retainer in the ion exchange membrane of the first conductivity type, and retaining an electrolyte; and an ion exchange membrane of the second conductivity type sandwiching the third electrolyte retainer with the ion exchange membrane of the first conductivity type, and selectively transmitting ions having an electric polarity that is the same as the electric polarity of the second electrode member. Accordingly, not only can the skin to be in contact with the non-operation electrode structure be prevented from burns and inflammation, but also the drug ions can be applied while stably supplying electricity to the skin. This enables the drug ions to be applied to the living organism securely and efficiently.

The summary does not necessarily describe all necessary features of certain embodiments of the present invention. The present invention may also be a sub-combination of the features described above.

### BRIEF DESCRIPTION OF THE DRAWINGS

FIG. 1 shows a top view of a packaged iontophoresis system 15.

FIG. 2 is a sectional view of the cross section of a-a′ line of FIG. 1, viewed from the right-hand side in FIG. 1.

FIG. 3 is a sectional view of the cross section of line b-b′ of FIG. 1, viewed from the bottom side in FIG. 1.

FIG. 4 is a sectional view of the cross section of line b-b′ of FIG. 1, after the opening and removal of a package material 80, viewed from the bottom side in FIG. 1.

FIG. 5 is a bottom view after the opening and removal of the package material 80, viewed from the rear side in FIG. 1.

FIG. 6 is a top view of a packaged iontophoresis system 25.

FIG. 7 is a top view of a packaged iontophoresis system 35.

FIG. 8 is a schematic side view of an iontophoresis system 10.

### DESCRIPTION OF EXEMPLARY EMBODIMENTS

Some aspects of the invention will now be described based on the embodiments, which do not intend to limit the scope of the present invention, but rather, exemplify the invention. All of the features and the combinations thereof described in the embodiment are not necessarily essential to the invention.

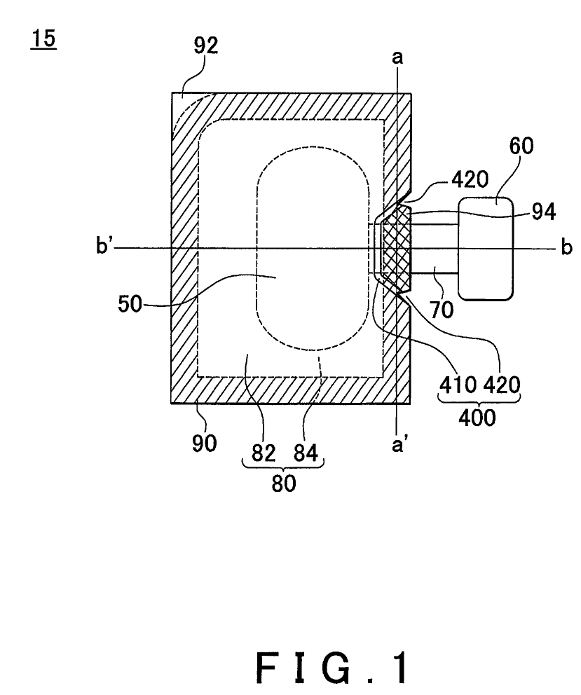

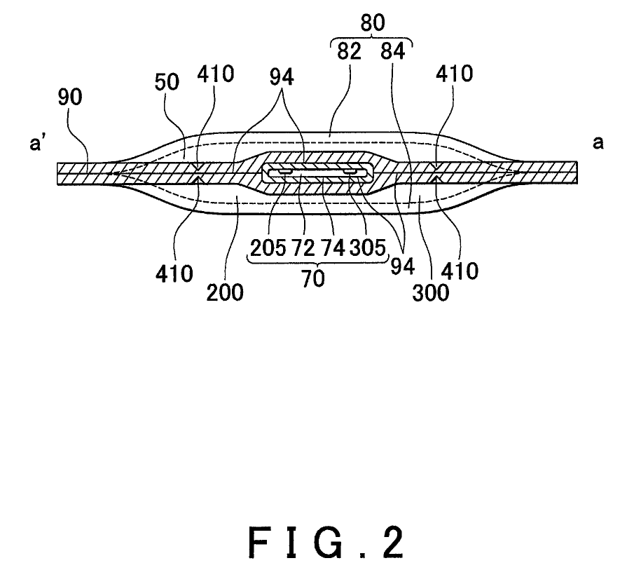

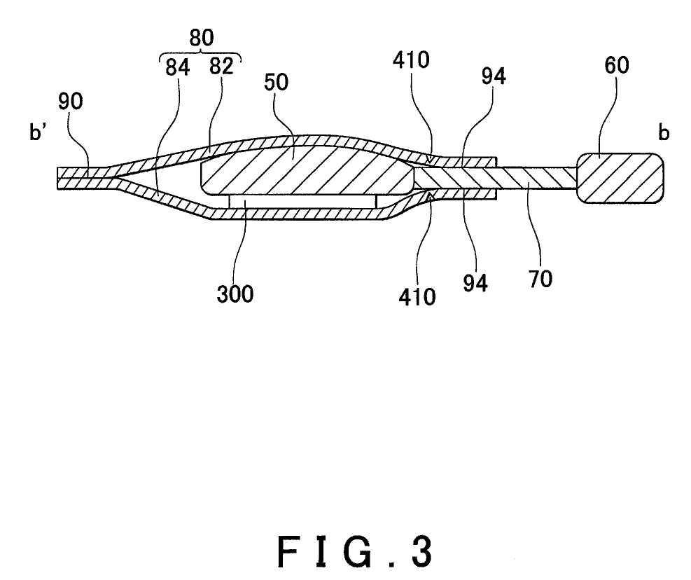

FIG. 1 shows a top view of a packaged iontophoresis system 15. FIG. 2 is a sectional view of the cross section of a-a′ line of FIG. 1, viewed from the right-hand side in FIG. 1. FIG. 3 is a sectional view of the cross section of line b-b′ of FIG. 1, viewed from the bottom side in FIG. 1. As in FIG. 1, the packaged iontophoresis system 15 includes an iontophoresis system 10 and a package material 80, where the iontophoresis system 10 includes an apparatus body 50, a power source 60, and a connection part 70 for connecting the apparatus body 50 to the power source 60, and the package material 80 is formed substantially as a bag shape by welding of an upper package material 82 and a lower package material 84 at a welded surface 90 and a connection-part welded surface 94.

It is preferable that the upper package material 82 and the lower package material 84 are water-impermeable and further substantially impermeable to gases body such as water vapor. The material may accordingly be an aluminum foil, a polyester film, a polypropylene film, a polyethylene film, or the like. When being welded by means of heat sealing, the upper package material 82 and the lower package material 84 may be a lamination of a plurality of the above-described films, or may be the above-described films with a polymer resin coating for facilitating peeling at the time of opening.

As in FIG. 2, the connection part 70 includes a substrate 72 forming an active conductive wire 205 and a counter conductive wire 305 thereon, and a coating material 74 which is provided in the circumference of the cross section of the substrate 72. The substrate 72 is preferably a material having electrical insulation properties and flexibility, such as a polyimide film. An exemplary method of forming the active conductive wire 205 and the counter conductive wire 305 on the surface of the substrate 72 is to deposit or to photo-etch a conductive material such as copper on the surface of the substrate 72. It is preferable that the coating material 74 is made of the same material as the upper package material 82 and the lower package material 84, or is made of a material which is easily welded to the upper package material 82 and the lower package material 84.

As shown in FIG. 1 through FIG. 3, the upper package material 82 and the lower package material 84 are welded to the connection part 70 without any gap therebetween, to sandwich the connection part 70 from above and below, at the connection part welded surface 94. As a result, the apparatus body 50 of the iontophoresis system 10, and the portion of the connection part 70 positioned nearer to the apparatus body 50 than to the connection-part welded surface 94, is hermetically sealed inside the package material 80.

The method of fixing the upper package material 82 and the lower package material 84 is not limited to welding by heat sealing described above. Other alternative methods include attachment by means of a variety of adhesives, mechanical pressure bonding by means of clips and the like, or a combination thereof. Since the packaged iontophoresis system 15 is packed by the package material 80, the positioned nearer to the apparatus body 50 is more hermetically sealed than the apparatus body 50 of the iontophoresis system 10 and the connection-part welded surface 94 of the connection part 70. Decreases in the amount of ionic drugs or drug-dissolving solvent due to volatilization, and the corrosion of the power source 60 attributable to the vaporized drug due to the volatilization, are thus prevented.

Also as shown in FIGS. 1 through 3, the package material 80 includes a cutting guide 400 comprising a groove 410 and a notch 420. The groove 410 is for example formed up to half of the thickness from the respective surfaces of the upper package material 82 and the lower package material 84 in the position between the welded surface 90 and the connection-part welded surface 94. Also shown in FIG. 1, the groove 410 is formed to surround the connection-part welded surface 94 on the respective surfaces of the upper package material 82 and the lower package material 84. At the end thereof, the notch 420 is provided to protrude from the upper package material 82 and the lower package material 84 in the thickness direction. Further as shown in FIGS. 1 and 2, the package material 80 includes a non-welded surface 92 adjacent to the welded surface 90.

In the packaged iontophoresis system 15 of FIG. 1, when applying iontophoresis, first, the upper package material 82 is pulled from the non-welded surface 92 towards the upper direction in FIG. 2. During this operation, the upper package material 82 is cut at the cutting guide 400, and the portion of the upper package material 82 surrounded by the cutting guide 400 in FIG. 1 remains welded with the connection part 70. Next, the lower package material 84 is cut from the notch 420 along the groove 410.

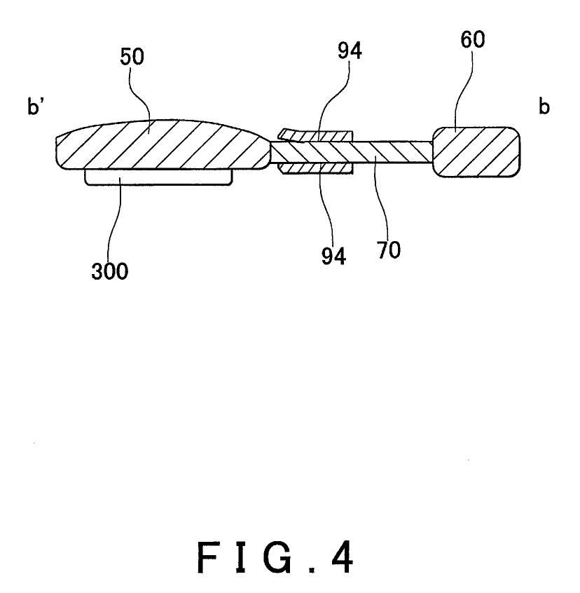

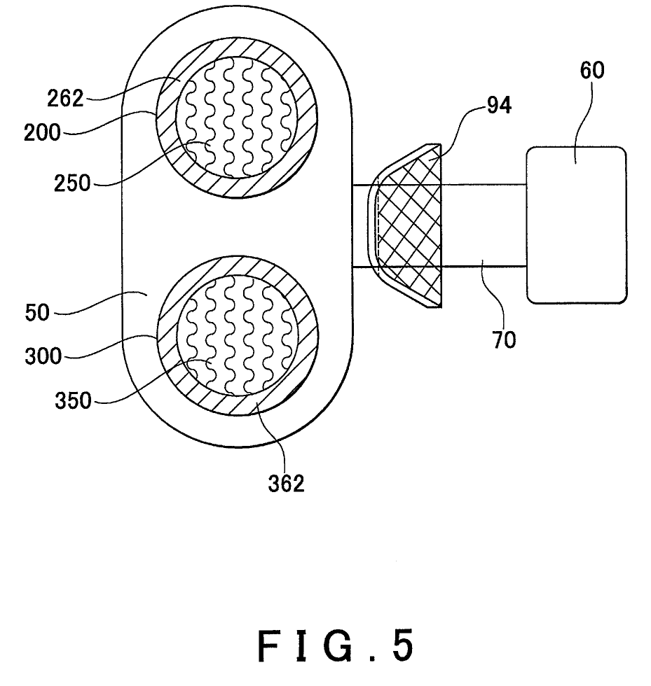

FIG. 4 is a sectional view of the cross section of line b-b′ of FIG. 1, after the opening and removal of a package material 80, viewed from the bottom side in FIG. 1. FIG. 5 is a bottom view after the opening and removal of the package material 80, viewed from the rear side in FIG. 1. As shown in FIGS. 4 and 5, the package material 80 is cut along the groove 410 of the cutting guide 400, and the iontophoresis system 10 is cut off therefrom with the mentioned opening operation. Consequently, the connection-part welded surface 94 of the respective parts of the upper package material 82 and of the lower package material 84 and a part surrounded by the cutting guide 400 are cut off from the package material 80 to remain welded with the connection part 70.

In this way, the cutting guide 400 guides the cutting line so that the part welded to the connection part 70 remains at the connection part 70. Therefore, the package material 80 can be opened and removed without peeling off the connection-part welded surface 94. As a result, the package material 80 is opened and removed without damaging the active conductive wire 205 and the counter conductive wire 305 of the connection part 70. The welding strength for the connection-part welded surface 94 may be stronger than the welding strength for the welded surface 90. In this case, the connection-part welded surface 94 will be more difficult to peel off when opening and removing the package material 80, to further facilitate opening and removal of the package material 80 without damaging the active conductive wire 205 and the counter conductive wire 305 of the connection part 70.

As shown in FIG. 5, after the opening and removal of the package material 80, a first conductive type ion exchange membrane 250, serving as a contacting surface with skin in the operation electrode structure 200 of the iontophoresis system 10, and an adhesive surface 262 of active side having adherence property are exposed. Likewise, a second conductive type ion exchange membrane 350, serving as a contacting surface with skin in the non-operation electrode structure 300 of the iontophoresis system 10, and an adhesive surface 362 of counter side having adherence property are exposed. In application of iontophoresis, the first conductive type ion exchange membrane 250 and the active adhesive surface 262 are maintained by adherence to be in contact with the skin of the living body at the dosing target part, while maintaining the second conductive type ion exchange membrane 350 and the counter side adhesive surface 362 to be in contact with the part surrounding the dosing target part or being adjacent to the dosing target part. Herein, the first conductive type represents one electric polarity of positive or negative, and the second conductive type represents the other electric polarity.

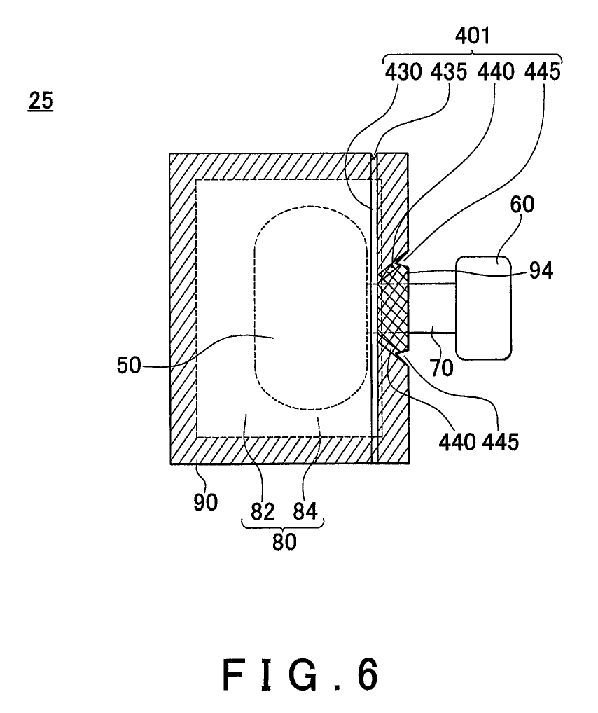

FIG. 6 is a top view of a packaged iontophoresis system 25, another embodiment of an iontophoresis system. The parts of the packaged iontophoresis system 25 in FIG. 6 assigned the same reference numerals have the same configurations as those of the packaged iontophoresis system 15 in FIG. 1, and so the explanation thereof is omitted below. As shown in FIG. 6, the package material 80 of the packaged iontophoresis system 25 includes a cutting guide 401 comprising a groove 430, a notch 435, a groove 440, and a notch 445. That is, the upper package material 82 and the lower package material 84 respectively include a cutting guide 401 in symmetric positions with each other, and the notch 435 and the notch 445 are provided to protrude the upper package material 82 and the lower package material 84 in the thickness direction respectively.

To use the packaged iontophoresis system 25 of FIG. 6 for iontophoresis, first, the package material 80 is cut from the notch 435 along the groove 430, thereby cutting off the package material 80 positioned at the left-hand side of the groove 430 in FIG. 6 from the iontophoresis system 10. During this operation, the package material 80 positioned at the right-hand side of the groove 430 in FIG. 6 remains welded with the connection part 70. Next, the remaining part is cut off from two notches 445 respectively along the groove 440 shown in FIG. 6. According to the above-described operation, the packaged iontophoresis system 25 is opened, while the parts respectively of the upper package material 82 and the lower package material 84 surrounded by the groove 430 and the groove 440 remain welded with the connection part 70.

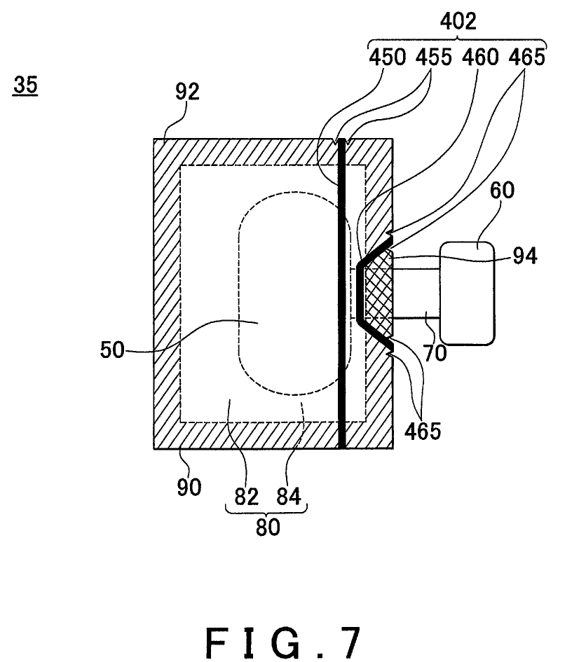

FIG. 7 is a top view of a packaged iontophoresis system 35, being further different embodiment. The parts of the packaged iontophoresis system 35 in FIG. 7 assigned the same reference numerals have the same configurations as those of the packaged iontophoresis system 15 in FIG. 1, and so the explanation thereof is omitted in the following. As shown in FIG. 7, the package material 80 of the packaged iontophoresis system 35 includes a cutting guide 402 comprising a tape 450, a notch 455, a tape 460, and a notch 465. The upper package material 82 and the lower package material 84 respectively include a cutting guide 402 in symmetric positions with each other, and the notch 455 and the notch 465 are provided to protrude the upper package material 82 and the lower package material 84 in the thickness direction respectively.

To use the packaged iontophoresis system 35 FIG. 7 for iontophoresis, first, the package material 80 is cut from the notch 455 along the tape 450, thereby cutting off the package material 80 positioned at the left-hand side of the tape 450 in FIG. 7 from the iontophoresis system 10. During this operation, the package material 80 positioned at the right-hand side of the tape 450 in FIG. 7 remains welded with the connection part 70. Next, the remaining part is cut off from the notch 465 shown in FIG. 7, respectively along the tape 460. According to the above-described operation, while the packaged iontophoresis system 35 is opened, the parts in the upper package material 82 and the lower package material 84 surrounded by the tape 460 remain welded with the connection part 70.

The tape 450 (460) of the packaged iontophoresis system 35 in FIG. 7 is not limited as long as it can guide the cutting line when cutting the package material 80 along the tape 450 (460) from the notch 455 (465), but a stretch film tape attached to the package material 80 may be included. It is desirable that each tape 450 (460) use a reel of tape wound around the upper package material 82 and the lower package material 84 of the package material 80 starting from the notch 455 (465) respectively.

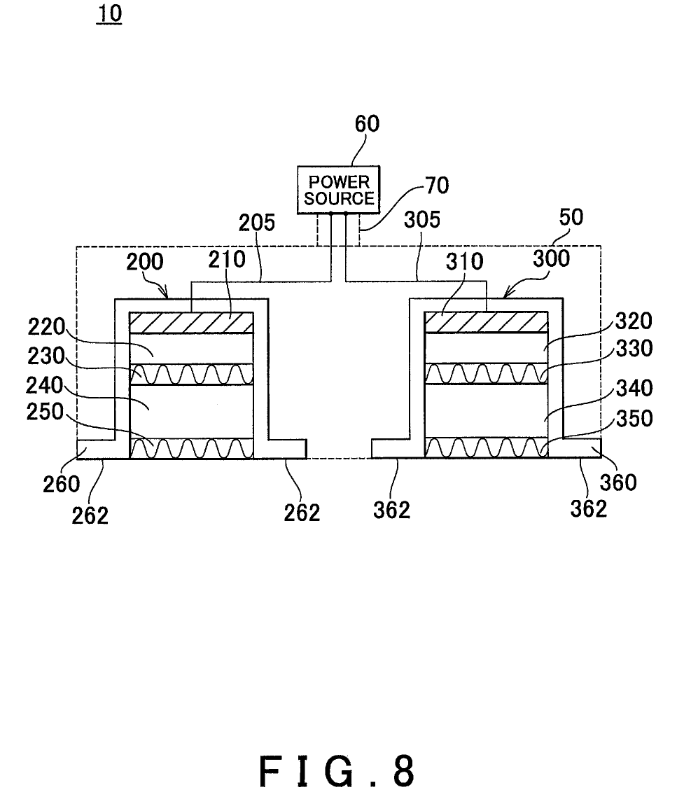

FIG. 8 is a schematic side view of an iontophoresis system 10. As shown in FIG. 8, the operation electrode structure 200 includes a first electrode member 210, a first electrolyte retainer 220, an ion exchange membrane 230 of a second conductive type, a drug solution retainer 240, and an ion exchange membrane 250 of a first conductive type in the stated order in the direction from the apparatus body 50 to the skin to be contacted to the iontophoresis system 10. The upper and side surfaces thereof are covered by a container 260. The first electrode member 210 is electrically connected to a terminal of the first conductivity type of the power source 60, by means of the active conductor 205.

The first electrolyte retainer 220 is electrically connected to the first electrode member 210, and retains an electrolyte. A compound having an oxidation reduction potential lower than that of water when compared to electrolysis reaction of water (i.e. oxidation and reduction of water) and that is easily oxidized and reduced is dissolved in the electrolyte. The ion exchange membrane 230 of the second conductivity type sandwiches the first electrolyte retainer 220 with the first electrode member 210, and selectively permeates ions of the second conductivity type. The drug solution retainer 240 retains drug solutions containing drug ions. Drug ions are either anions or cations resulting from ionic dissociation of drugs, and are ions of the first conductivity type having drug efficacy. The ion exchange membrane 250 of the first conductivity type sandwiches the drug solution retainer 240 with the ion exchange membrane 230 of the second conductivity type, and selectively permeates ions of the first conductivity type.

As shown in FIG. 8, the non-operation electrode structure 300 includes a second electrode member 310, a second electrolyte retainer 320, an ion exchange membrane 330 of a first conductive type, a third electrolyte retainer 340, and an ion exchange membrane 350 of a second conductive type in the stated order in the direction from the apparatus body 50 to the skin to be contacted to the iontophoresis system 10. The second electrode member 310 is electrically connected to a terminal of the second conductivity type of the power source 60, by means of the counter conductor 305.

The second electrolyte retainer 320 is electrically connected to the second electrode member 310, and retains an electrolyte. The ion exchange membrane 330 of the first conductivity type sandwiches the second electrolyte retainer 320 with the second electrode member 310, and selectively permeates ions of the first conductivity type. The third electrolyte retainer 340 is arranged in the opposite side of the second electrolyte retainer 320 against the ion exchange membrane 330 of the first conductivity type, and retains an electrolyte. The ion exchange membrane 350 of the second conductivity type sandwiches the third electrolyte retainer 340 with the ion exchange membrane 330 of the first conductivity type, and selectively permeates ions of the second conductivity type. Note that a compound having an oxidation reduction potential lower than the oxidation reduction potential of water when compared to an electrolysis reaction of water (i.e. oxidation and reduction of water) and that is easily oxidized and reduced is dissolved in the electrolyte retained by the second electrolyte retainer 320 and in the third electrolyte retainer 340, similar to the electrolyte retained by the first electrolyte retainer 220 of the operation electrode structure 200.

The container 260 of the operation electrode structure 200 and the container 360 of the non-operation electrode structure 300 include, at the contact surface thereof, an active adhesive surface 262 and a counter adhesive surface 362 having adherence property respectively. The active adhesive surface 262 and the counter adhesive surface 362 are shown in FIG. 5, and are maintained to be in contact with the skin, to adhere to and retain the operation electrode structure 200 and the non-operation electrode structure 300.

When power is supplied, i.e. when voltage is applied, to the first electrode member 210 and the second electrode member 310 from the power source 60 while the operation electrode structure 200 and the non-operation electrode structure 300 of the iontophoresis system 10 are in contact with the skin, electric current runs between the first electrode member 210 and the second electrode member 310 with the skin therebetween, effecting iontophoresis.

Here, the concrete configuration of the iontophoresis system 10 is detailed by taking an example where the drug ions are anions. In this example, the first conductivity type is negative, and the second conductivity type is positive. Therefore, the first electrode member 210 of the operation electrode structure 200 is a cathode, and the second electrode member 310 of the non-operation electrode structure 300 is an anode. In addition, in the operation electrode structure 200, the ion exchange membrane 230 of the second conductivity type is a cation exchange membrane, and the ion exchange membrane 250 of the first conductivity type is an anion exchange membrane. Further, in the operation electrode structure 200, the ion exchange membrane 330 of the first conductivity type is an anion exchange membrane, and the ion exchange membrane 350 of the second conductivity type is a cation exchange membrane.

The iontophoresis system 10, during usage, has the following advantages. In the operation electrode structure 200, the drug ions included in the drug solution retained by the drug solution retainer 240 moves to the opposite side (to the side of the skin) of the first electrode member 210 being the cathode, by means of the electrophoresis. Then the drug ions are permeated to the skin from the drug solution retainer 240 through the ion exchange membrane 250 of the first conductivity type contacting the skin. On the other hand, cations in the living body are not permeated through the ion exchange membrane 250 of the first conductivity type, and do not move toward the drug solution retainer 240. Therefore, the drug ions are able to be introduced to a living body by iontophoresis in a stable use state. Cations in the drug solution retainer 240 pairing with the drug ions (anion) move toward the first electrode member 210, are permeated through the ion exchange membrane 230 of the second conductivity type being a cation exchange membrane, and move towards the first electrolyte retainer 220. Consequently, ionic balance is maintained in the drug solution retainer 240 in the use state, and so the pH change hardly occurs. As a result, the energizing resistance does not substantially increase, restraining reductions in transportation efficiency of the drug ions.

On the other hand, in the non-operation electrode structure 300, a compound dissolved in the electrolyte retained by the third electrolyte retainer 340 has an oxidation reduction potential lower than that of water. Therefore, electrolysis of water in the second electrode member 310 acting as an anode does not occur. Consequently, an increase of energizing resistance is prevented because of prevention of the contact between the second electrode member 310 and the electrolyte retained in the third electrolyte retainer 340 by air bubbles (oxygen gas) generated due to electrolysis.

Note that when the drug ions are cations, the first conductivity type is positive, and the second conductivity type is negative. Therefore, the electric polarity of the first electrode member 210 and the second electrode member 310 are reversed in the iontophoresis system 10 shown in FIG. 8, and the types (ion selective characteristic) of the ion exchange membrane 230 of the second conductivity type, the ion exchange membrane 250 of the first conductivity type, the ion exchange membrane 330 of the first conductivity type, and the ion exchange membrane 350 of the second conductivity type are also reversed.

In the present embodiment, an exemplary drug ions provided for use with application of iontophoresis is as follows. An exemplary positively charged drug ions includes anesthetic (e.g. procaine hydrochloride and lidocaine hydrochloride), a therapeutic agent for gastrointestinal disease (e.g. carnitine chloride), a muscle relaxant suxametonium (e.g. pancronium bromide), and antibiotics (e.g. tetracyclines drug product, kanamycins drug product, and gentamicins drug product). An exemplary negatively charged drug ions includes a vitamin (hereinafter abbreviated as “V”) preparation (e.g. VB₂, VB₁₂, VC, VE, and folate), adrenal cortex hormone (e.g. water-soluble hydrocortisone drug product, water-soluble dexamethasone drug product, and water-soluble prednisolone drug product), and antibiotics (e.g. water-soluble penicillin drug product, and water-soluble chloramphenicol drug product).

Exemplary voltage used in application of iontophoresis is a pulse voltage for use as a low-frequency therapy equipment. The voltage may be gradually raised or lowered. The electric current running in a body is increased or decreased as necessary depending on the area of the first electrode member 210 and the second electrode member 310, the part of the body being the dosing target, or the individual difference of a person being the dosing target, so that the person would not suffer from pain or heat.

The power source 60 may constantly apply the voltage for applying the iontophoresis, to the first electrode member 210 and the second electrode member 310. However, there may be further provided means for detecting contact between the apparatus body 50 and the skin, to apply a voltage for applying the iontophoresis only when the apparatus body 50 is brought in contact with the skin. The power source 60 may be desorbable with respect to the connection part 70, or be integrally formed with the connection part 70. Concrete appropriate examples of the power source 60 include a battery, a constant-voltage device, a constant-current device, a constant-voltage/constant-current device (Galvanic device). It is desirable that the power source 60 has a favorable portability.

The electrode material of the first electrode member 210 and of the second electrode member 310 may be selected as necessary depending on the characteristics of the drug ions, and may be a conductive material such as carbon and platinum.

The first electrolyte retainer 220 of the operation electrode structure 200 and the second electrolyte retainer 320 and the third electrolyte retainer 340 of the non-operation electrode structure 300 preferably retain an electrolyte that is a solution in which a compound having an oxidation reduction potential lower than that of water compared to an electrolysis reaction of water (i.e. oxidation and reduction of water), and that is easily oxidized and reduced, is dissolved. For example, a mixed aqueous solution of first copperas (FeSO₄) and second copperas (Fe₂(SO₄)₃), an aqueous solution of sodium ascorbate, and a mixture aqueous solution of lactic acid and sodium fumarate. Furthermore, the retaining form of these electrolytes may be a gel, a desirable medium (gauze or water-absorbing polymer material) into which the electrolyte is impregnated, or a solution that retains the electrolyte as is.

For example, the anion exchange membrane may be a polymer having a quaternized ammonium group at the side chain thereof, and the cation exchange membrane may be a polymer having a sulfonate group at the side chain thereof. They may be selected desirable, and also be combined as appropriated depending on the type of the drug ions used.

It is preferable that the container 260 and the container 360 are made of a material that is an electric insulator and resistant to ionic conductivity, and has plasticity, bendability, or flexibility, and has a form-maintaining nature. Exemplary materials include acrylic, polyvinyl chloride, polyacrylic, polyamide, polysulphone, polystyrene, polyoxymethylene, polycarbonate, polyester, and copolymers thereof.

As stated above, with the present embodiment the package material 80 may be opened without peeling off the connection-part welded surface 94. Therefore, opening and removal of the package material 80 does not damage the active conductor 205 or the counter conductor 305 of the connection part 70.

Although some aspects of the present invention have been described by way of exemplary embodiments, the scope of the present invention is not limited thereto, and it should be understood that those skilled in the art might make many changes and substitutions without departing from the spirit and the scope of the present invention which is defined only by the appended claims.

As clear from the foregoing, according to one embodiment of the present invention, the decrease in ionic drugs or drug-dissolving solvent due to volatilization, and the corrosion of the power source attributable to the vaporized drug due to the volatilization are thus prevented.

What is claimed is:

1. A packaged iontophoresis system comprising:
an iontophoresis system including an apparatus body for transdermally delivering an ionized drug by iontophoresis, and a connection part extended from the apparatus body and connected to the apparatus body and a power source for supplying power to the apparatus body;
a package material hermetically packaging the apparatus body and a portion of the connection part, by at least a portion thereof being fixed to the connection part; and
a cutting guide guiding a cutting line of the package material so that the portion of the package material fixed to the connection part remains at the connection part when the package material is opened and removed.

2. The packaged iontophoresis system according to claim 1, wherein
the cutting guide includes a groove in a thickness direction and provided between the part thereof that is fixed to the connection part and the other part thereof.

3. The packaged iontophoresis system according to claim 2, wherein
the cutting guide includes a notch provided in a vicinity of the part thereof that is fixed to the connection part.

4. The packaged iontophoresis system according to claim 2, wherein
the part of the package material fixed to the connection part is fixed more strongly than any other part of the package material.

5. The packaged iontophoresis system according to claim 3, wherein
the part of the package material fixed to the connection part is fixed more strongly than any other part of the package material.

6. The packaged iontophoresis system according to claim 1, wherein
the cutting guide includes a notch provided in a vicinity of the part thereof that is fixed to the connection part.

7. The packaged iontophoresis system according to claim 6, wherein
the part of the package material fixed to the connection part is fixed more strongly than any other part of the package material.

8. The packaged iontophoresis system according to claim 1, wherein
the part of the package material fixed to the connection part is fixed more strongly than any other part of the package material.

9. The packaged iontophoresis system according to claim 1, wherein
the apparatus body includes:
an operation electrode structure including a first electrode member and a drug solution retainer, the first electrode member being electrically connected to a first conductivity type terminal of a power source, the first conductivity type being the same as a conductivity type of the ionized drug, the drug solution retainer retaining a drug solution containing the drug and being provided in an electric field created by the first electrode member; and
a non-operation electrode structure electrically connected to a second conductivity type terminal of the power source, the second conductivity type being opposite to the first conductivity type.

10. The packaged iontophoresis system according to claim 9, wherein
the operation electrode structure further includes:
a first electrolyte retainer electrically connected to the first electrode member and retaining an electrolyte;
an ion exchange membrane of the second conductivity type sandwiching the first electrolyte retainer with the first electrode member, and selectively permeating ions of the second conductivity type; and
an ion exchange membrane of the first conductivity type sandwiching the drug solution retainer with the ion exchange membrane of the second conductivity type, and selectively permeating ions of the first conductivity type.

11. The packaged iontophoresis system according to claim 10, wherein
the counter electrode structure includes:
a second electrode member electrically connected to the terminal of the second conductivity type of the power source;
a second electrolyte retainer electrically connected to the second electrode member and retaining an electrolyte;
an ion exchange membrane of the first conductivity type sandwiching the second electrolyte retainer with the second electrode member, and selectively permeating an ion having an electric polarity different from an electric polarity of the second electrode member;
a third electrolyte retainer provided in an opposite side of the second electrolyte retainer in the ion exchange membrane of the first conductivity type, and retaining an electrolyte; and
an ion exchange membrane of the second conductivity type sandwiching the third electrolyte retainer with the ion exchange membrane of the first conductivity type, and selectively permeating an ion having an electric polarity that is the same as the electric polarity of the second electrode member.
